(19) United States
(12) Patent Application Publication
(10) Pub. No.: US 2024/0358973 A1
(43) Pub. Date: Oct. 31, 2024

(54) PORT PROTECTOR AND COVER SYSTEM AND METHOD OF USE

(71) Applicant: Marsco Medical, LLC, Parkville, MO (US)

(72) Inventors: Scott A. Crain, Kearney, MO (US); Mark Jansen, Parkville, MO (US)

(21) Appl. No.: 18/764,301

(22) Filed: Jul. 4, 2024

Related U.S. Application Data
(63) Continuation-in-part of application No. 17/521,949, filed on Nov. 9, 2021.
(60) Provisional application No. 63/110,698, filed on Nov. 6, 2020.

(51) Int. Cl. A61M 25/02

(52) U.S. Cl. CPC A61M 25/02; A61M 2025/0246; A61M 2025/0253

(57) ABSTRACT

A protection device for a porta catheter device installed into patient's body. The protection device has a flat rim which would be secured to the patient's body around the porta catheter installation site using a medically appropriate adhesive. A dome-shaped protector rises up from the flat rim. The dome-shaped protector can secure any creams or lidocaine applied to the site of the porta catheter. The smooth round shape of the dome-shaped protector prevents it from catching or bumping on clothing or objects in the environment, preventing painful snags. There are no holes or gaps in the protector to prevent leaking of the creams or lidocaine. The flat rim can be affixed about the porta catheter using adhesive or a magnet.

### CROSS-REFERENCE TO RELATED APPLICATION

This application is a Continuation-in-Part of U.S. Non-Provisional patent application Ser. No. 17/521,949 Filed Nov. 8, 2021, which claims priority in U.S. Provisional Patent Application No. 63/110,698 Filed Nov. 6, 2020, all of which are incorporated herein by reference.

### BACKGROUND OF THE INVENTION

### 1. Field of the Invention

The present invention relates generally to a port protector and cover system and method for use thereof, and more specifically to a cover and protector for port a catheter devices installed in patients which serve to prevent creams and lidocaine from leaking away from the porta catheter site and to prevent pain and discomfort associated with bumping or snagging objects and clothing.

### 2. Description of the Related Art

Porta catheters, also called a port-a-cath or simply a port, is a device used to draw blood and deliver treatments to patients, such as cancer patients, including intravenous fluids, drugs, blood transfusions, or chemotherapy treatments. The port is placed under the skin, usually in the chest. It is attached to a catheter (a thin, flexible tube) that is guided (threaded) into a large vein above the right side of the heart called the superior vena cava. A port-a-cath may stay in place for many weeks or months. A needle is inserted through the skin into the port to draw blood or give fluids.

As these porta catheters are inserted into what is essentially an open wound in the body, they can be painful and are prone to snagging and bumping into objects and clothing. Patients can place creams or lidocaine about the site of the port to help with pain and discomfort. However, this can lead to a mess without properly covering the sit

Heretofore there has not been available a system or method for a porta catheter protector with the advantages and features of the present invention.

### BRIEF SUMMARY OF THE INVENTION

The present invention generally provides a protection device for a porta catheter device installed into patient's body. The protection device has a flat rim which would be secured to the patient's body around the porta catheter installation site using a medically appropriate adhesive. A dome-shaped protector rises up from the flat rim. The dome-shaped protector can secure any creams or lidocaine applied to the site of the porta catheter. The smooth round shape of the dome-shaped protector prevents it from catching or bumping on clothing or objects in the environment, preventing painful snags. There are no holes or gaps in the protector to prevent leaking of the creams or lidocaine.

The protector device can be placed on the patient's body using double-sided tape or some other suitable adhesive. An embodiment could also include a magnet for magnetically securing the protector to the access port.

### BRIEF DESCRIPTION OF THE DRAWINGS

The drawings constitute a part of this specification and include exemplary embodiments of the present invention illustrating various objects and features thereof.

FIG. 1 is a three-dimensional isometric view of a preferred embodiment of the present invention.

FIG. 2 is a partially-exploded three-dimensional isometric view thereof shown from below.

FIG. 3 is a front elevational view showing the embodiment of FIG. 1 shown in a typical environment secured about a catheter port.

FIG. 4 is a top plan. thereof.

FIG. 5 is a bottom plan view thereof.

FIG. 6 is a side elevational view thereof.

FIG. 7 is a side sectional view taken about the line of FIG. 6.

FIG. 8 is a three-dimensional isometric view of an alternative embodiment of the present invention.

FIG. 9 is a partially-exploded three-dimensional isometric view thereof shown from below.

FIG. 10 is a side elevational view showing the embodiment of FIG. 7 shown in a typical environment secured about a catheter port.

FIG. 11 is a partially exploded three-dimensional isometric view showing an alternative embodiment of the present invention with an adhesive ring element being placed over a protective cover element.

FIG. 12 is a three-dimensional isometric view thereof, shown in an assembled orientation.

FIG. 13 is a side elevational view thereof.

FIG. 14 is a side sectional view taken about the line of FIG. 13.

FIG. 15 is a side elevational view showing the embodiment thereof in a typical environment being secured about a catheter port.

### DETAILED DESCRIPTION OF THE PREFERRED EMBODIMENTS

### I. Introduction and Environment

As required, detailed aspects of the present invention are disclosed herein, however, it is to be understood that the disclosed aspects are merely exemplary of the invention, which may be embodied in various forms. Therefore, specific structural and functional details disclosed herein are not to be interpreted as limiting, but merely as a basis for the claims and as a representative basis for teaching one skilled in the art how to variously employ the present invention in virtually any appropriately detailed structure.

Certain terminology will be used in the following description for convenience in reference only and will not be limiting. For example, up, down, front, back, right and left refer to the invention as orientated in the view being referred to. The words, “inwardly” and “outwardly” refer to directions toward and away from, respectively, the geometric center of the aspect being described and designated parts thereof. Forwardly and rearwardly are generally in reference to the direction of travel, if appropriate. Said terminology will include the words specifically mentioned, derivatives thereof and words of similar meaning.

### II. Preferred Embodiment Catheter Port Protector 2

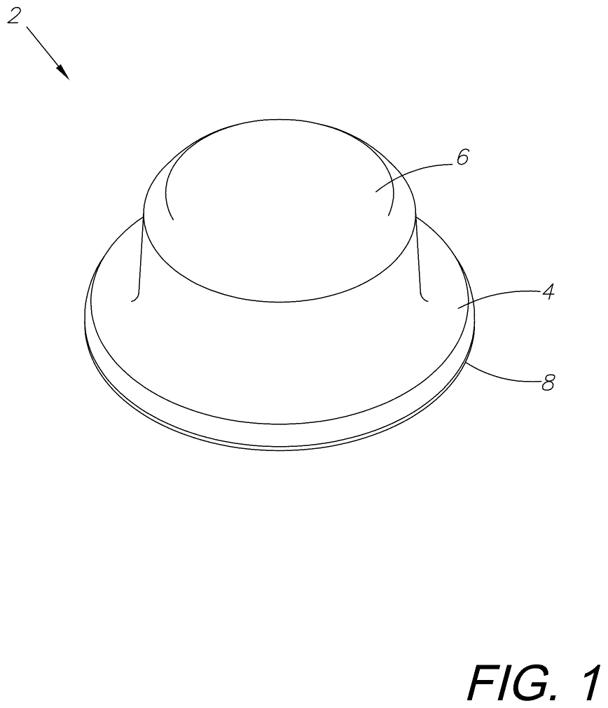

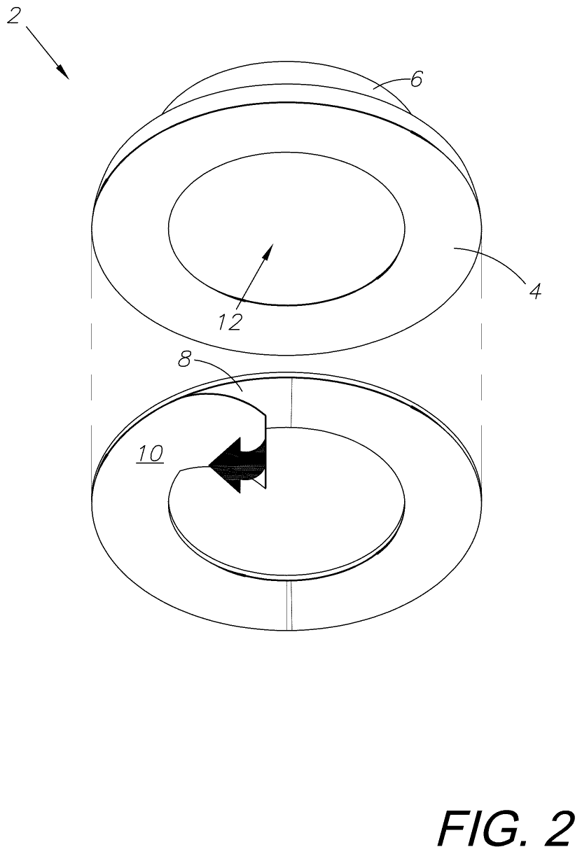

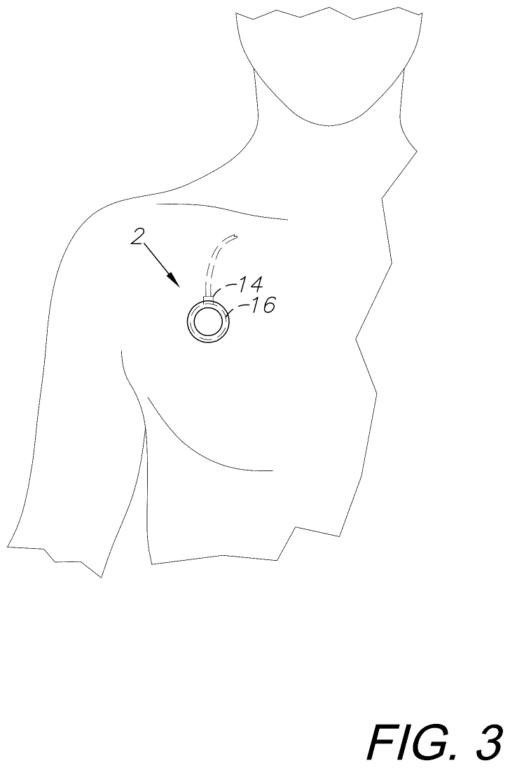

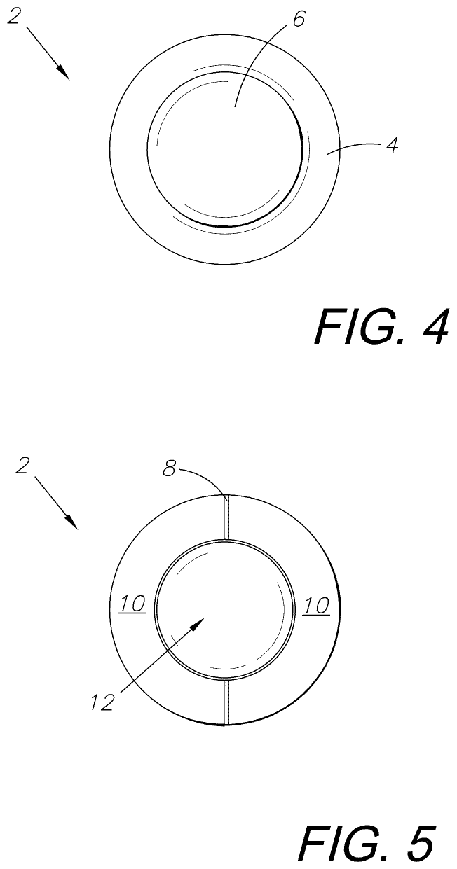

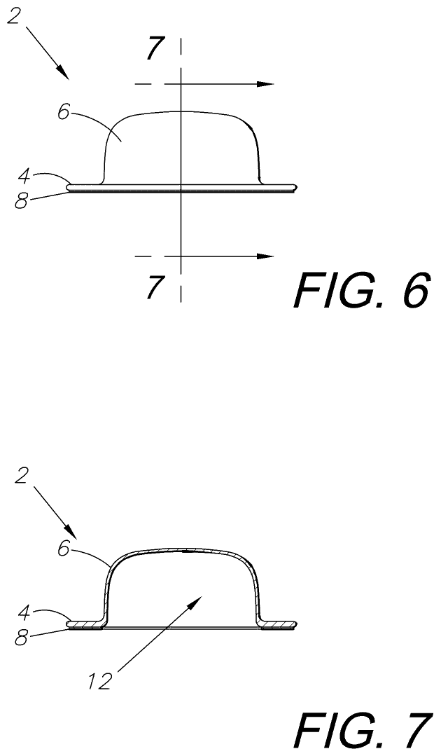

As shown in FIGS. 1-7, the present invention is a porta catheter 14 protector device 2 designed to both protect the porta catheter insertion site and access port 16 as well as prevent creams and lidocaine applied about the site from seeping or leaking out of the protector.

The protector device 2 has a flat rim 4 which would be adhered to the patient's body about the insertion site of the porta catheter 14 using a medically safe adhesive 8. The adhesive may have a removable layer 10 which can be removed when the protector is being placed. A dome 6 rises up away from the flat rim 4, forming an interior space 12 for protecting the access port 16 and which may contain the creams and/or lidocaine and protects the porta catheter device from snagging or bumping into objects or clothing.

The adhesive 8 could be a reusable adhesive or a double-sided tape disc which can be replaced as needed.

### III. Alternative Embodiment Catheter Port Protector 52

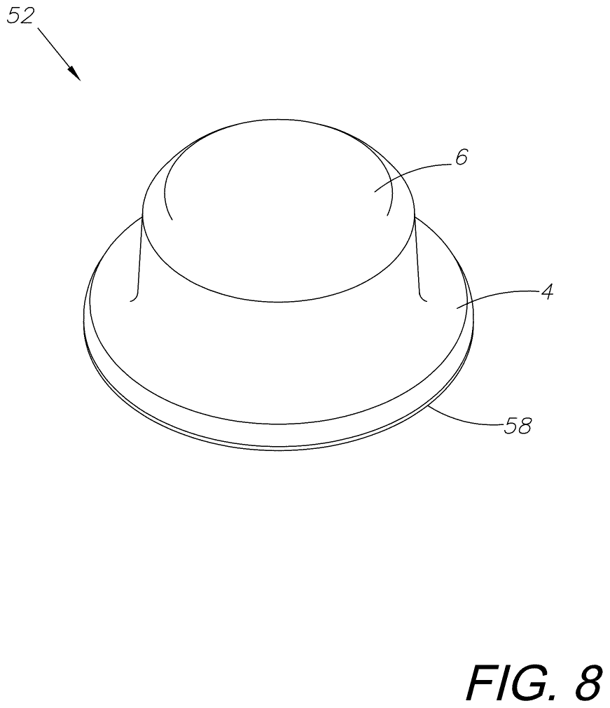

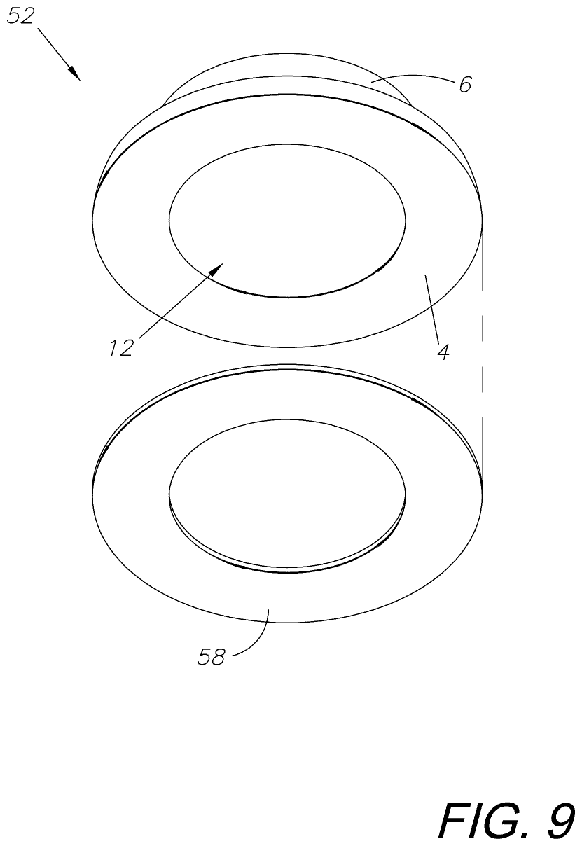

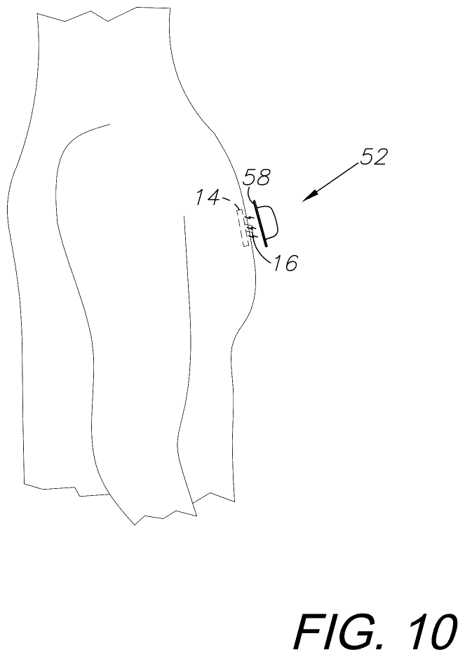

FIGS. 8-10 show a slightly alternative embodiment port protector device 52 which includes the same flat rim 4 and dome 6 forming an internal cavity 12. Instead of an adhesive, a magnetic ring 58 is affixed to the base of the flat rim 4. This magnet can magnetically connect to the catheter 14 and/or port 16, thereby surrounding and securing the access port 16 just like the previous embodiment.

### IV. Alternative Embodiment Catheter Port Protector 102

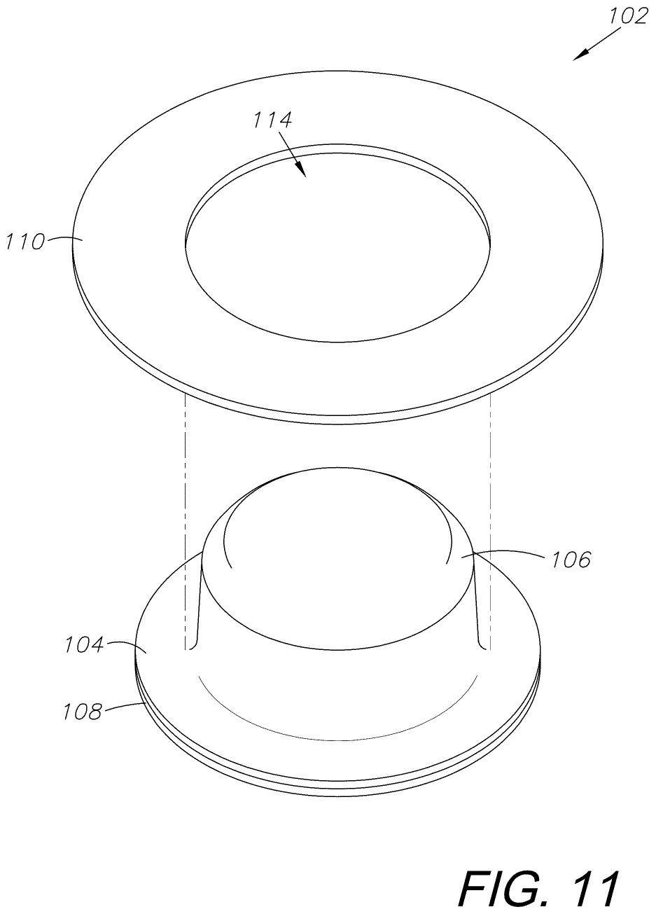

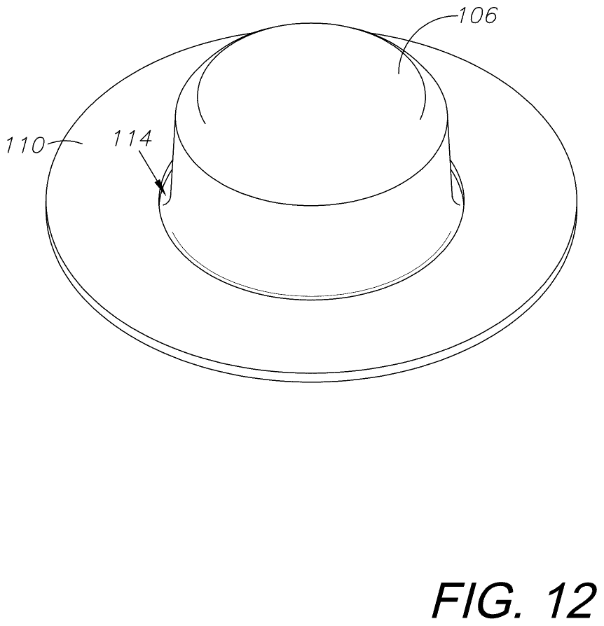

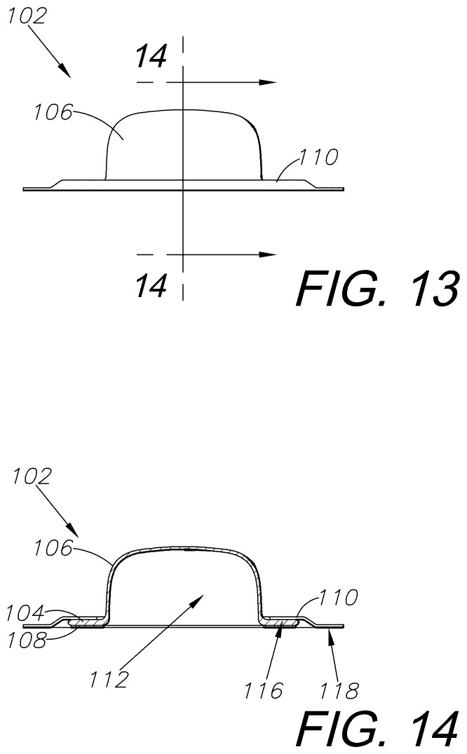

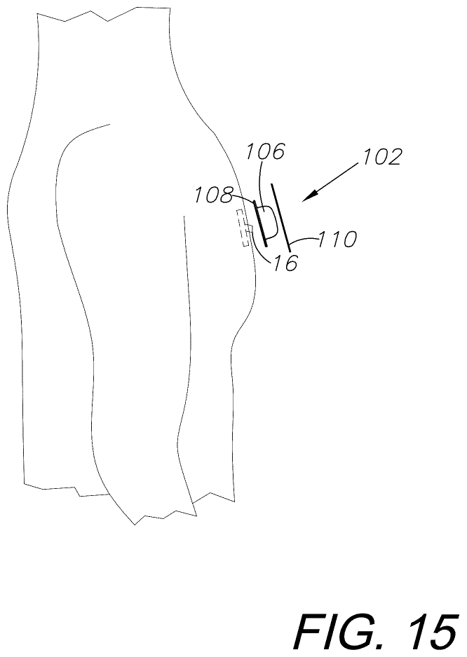

FIGS. 11-15 show another alternative embodiment catheter port protector device 102 which includes a flat rim 104 and dome 106 as before, with a larger rim optionally provided. An adhesive ring 110 can be placed over the flat rim 104 such that a portion of the adhesive ring 110 extends beyond the edge of the flat rim 104 and contacts the patient's body, thereby securing the port protector device 102 in place.

An optional padded ring 108 is shown affixed to the bottom face 116 of the flat rim 104. This could be affixed using adhesive or any other suitable method. The padded ring 108 offers additional comfort about the port 16 placement.

It is to be understood that while certain embodiments and/or aspects of the invention have been shown and described, the invention is not limited thereto and encompasses various other embodiments and aspects.

Having thus described the invention, what is claimed as new and desired to be secured by Letters Patent is:

1. A catheter port configured to be placed on the exterior of a body, thereby covering a port entering the body, the port protector comprising:
a protector body comprising a single uniform body including both a flat rim portion and a dome portion without separation between said flat rim portion and said dome portion, whereby said flat rim portion extends upwards away from said flat rim, forming an interior space;
said flat rim configured to be placed about a catheter insertion site and an associated access port on the exterior of the body at said catheter insertion site;
an adhesive ring comprising a hole configured to fit around said dome, such that a portion of said adhesive ring is secured about a top face of said flat rim and a portion of said adhesive ring is secured against the body about said catheter insertion site; and
said interior space configured to be placed around and over said access port with no portion of said protector body being placed within said body.

2. The catheter port protector of claim 1, further comprising a padded ring affixed to a bottom face of said flat rim, said padded ring sized to fit said bottom face of said flat rim.
